(19) United States
(12) Patent Application Publication
(10) Pub. No.: US 2017/0006826 A1
(43) Pub. Date: Jan. 12, 2017

(54) Livestock Management Apparatus and Method

(71) Applicant: Pablo Rafael Torres, Buenosaires (AR)

(72) Inventor: Pablo Rafael Torres, Buenosaires (AR)

(21) Appl. No.: 14/792,884

(22) Filed: Jul. 7, 2015

(51) Int. Cl. A01K 13/00; A61D 7/00; A61M 11/00; A01K 29/00; A01K 27/00

(52) U.S. Cl. CPC A01K 13/003; A01K 29/005; A01K 27/007; A61M 11/00; A61D 7/00

(57) ABSTRACT

An apparatus and method directionally administers at least one medication directly onto livestock while also monitoring the effect of the medication and tracking the movement and orientation of the livestock. The apparatus periodically administers the medication at a predetermined dosage. The apparatus also tracks the position and orientation of the livestock as an indication of whether the medication is effective. A solar cell provides renewable energy for powering the apparatus. A dispenser directionally and topically sprays medication on the wither area of the livestock. The medication is stored in at least one reservoir. A tracking portion tracks the position of the livestock in a predetermined range. The orientation of the livestock is also determined. The apparatus can be controlled directly from a control portion, or remotely from a remote processor. The apparatus minimize time and costs for managing livestock, especially over large areas and large numbers of livestock.

### BACKGROUND

Typically, livestock are domesticated animals raised in an agricultural setting to produce commodities such as food, fiber, and labor. Common types of livestock include, bovines, goats, sheep, chickens, and horses. Livestock are generally raised for profit, and thus are a valuable commodity to be protected against disease, sickness, and theft. The inventor was familiar with livestock, and in particular animal husbandry, which is a component of modern agriculture.

The inventor was aware that livestock are generally kept in an enclosure, are fed by human-provided food, and are intentionally bred. However, the inventor was also aware that some livestock are not enclosed, but rather, allowed to breed freely. The inventor knew that in large open ranges the livestock may be only occasionally inspected or yarded in round-ups for counting and assessing the health of the livestock. Often, herding dogs or cowboys could be used for mustering the livestock. But this could be costly, especially for ranches ranging thousands of acres. Nonetheless, the inventor knew that whether fenced up, or free to roam over wide ranges, livestock was susceptible to disease and health concerns.

The inventor observed livestock operations and observed some common problems. The largest problem seen by the inventor was that livestock diseases were difficult to monitor and cure, especially with many animals spread out over a large area. The inventor saw that livestock diseases from these pests compromise animal welfare, reduction of productivity, and potential infection of humans. However, it was soon apparent to the inventor that livestock diseases could be reduced through antibiotics and vaccines. The inventor was aware that several types of pests afflict range animals, especially herds of cattle, and caused substantial economic loss. Among these pests are horn flies, Haematobia irritans (L.); face flies, Musca autumnalis De Geer; the Gulf Coast tick, Amblyomma maculatum Koch; the screwworm, Cochliomyia hominivorax (Coquerel); and the southern cattle tick, Ripicephalus (Boophilus) microplus (Canestrini).

Through general observation, the inventor realized that to control these insects and pests, the livestock were treated by spraying their hair coats with an insecticide solution. However, it soon became evident that this type of treatment provides protection against the pests for a period of about three weeks. Beyond this period of time, the insecticide treatment is usually ineffective because the insecticide was degraded by light and microorganisms. In addition, the use of relatively large amounts of the insecticide is necessitated by the need to spray the entire surface of the animal and to compensate for the gradual deterioration of the insecticide following application to the animal.

The inventor was aware of another possible solution to treating the livestock. This involved livestock ear tags comprising a composite polymer with pesticide or repellent dispersed thereon or therein are known. However, the inventor soon realized that such devices suffer from various disadvantages, such as not being reusable; of having a release rate which is nonzero order or declining over time, promotes or raises the chances of resistance, nonadjustable and of relatively short duration; of insufficient tensile and ductile strength which contributes to brittleness, thus causing loss through breakage; and, in some cases, toxicity to the livestock. Thus, the inventor recognized that year round and unmonitored treatment of the livestock disease was preferable to the aforementioned solutions.

The inventor designed a periodic dispersing electromechanical device that periodically dispersed a predetermined dosage of medication. The idea was to automate the administering of treatment medicine directly on the livestock. However, the inventor soon realized that the battery would die quickly and negate all the advantages of an automated medication dispenser provided.

The inventor was aware that portable solar cell electricity chargers have been developed to receive and convert sunlight into electrical energy. The inventor also knew that livestock chiefly remained outside under the sun. The inventor decided to integrate a solar cell into the medication dispenser, such that it could operate for extended period of times (a year or longer) without battery replacement or power outages.

However, the device continued to be disturbed while attached to the animal. Other animals or fences, posts, and trees would knock it from its place on the animal. The inventor was aware that the wither area of the livestock provided the optimal area for absorption of medication into the bloodstream. The inventor also noticed that periodically spraying a small quantity of medication on the wither area of the livestock was more effective than dosing the animal with a large quantity over a longer period of time. The inventor also recognized that including a directional dispensing tube on the device would also help to direct the medication on the wither area as well as to the tail or head.

The inventor then modified the dispensing device by providing a remote processor that could operatively communicate with the device. And since the device was now operable remotely, the inventor decided to integrate a tracking processor and chip in the apparatus. In this way, the device could be remotely operated and the livestock could be remotely tracked. The inventor finally took the step of including a gyroscope to the tracking chip so that it would be apparent if an animal fell to its side, such as in an accident or death. Thus, the inventor created a livestock management method based on the dispensing device.

Managing livestock for medication and tracking purposes has been performed in the past, yet none with the present characteristics of the present invention. See Patent numbers: U.S. 20050241589; U.S. 20120012069; and WO2013085379.

For the foregoing reasons, there is a need for an apparatus and method for directionally administering medication onto livestock while also monitoring the effect of the medication, and tracking the movement and orientation of the livestock.

### SUMMARY

The present invention describes a livestock management apparatus and method for managing the welfare of livestock, and specifically the medication and location of at least one livestock. In some embodiments, the apparatus and method may directionally administer at least one medication directly onto at least one livestock while also monitoring the effect of the medication and tracking the movement and orientation of the livestock. The apparatus periodically administers the at least one medication at a predetermined dosage during a prolonged or extended period of time. The apparatus also tracks the position and orientation of the livestock as an indication of whether the medication is effective. The apparatus and method minimize time and costs associated with managing livestock, especially over large areas or with large numbers of livestock. The dosage amount and intervals are set by the operator either on site on the apparatus or remotely by radio, Wi-Fi or any other form of remote transmission.

In some embodiments, the apparatus and method provides a medication administration and tracking means that attaches directly to the livestock. The apparatus is the chief component for the periodic medical administration and tracking of the livestock. The apparatus utilizes at least one strap to attach proximally to the withers of the livestock in a disposition that minimizes movement and slippage by the apparatus, but also optimizes directional administration of the medication. In one embodiment, the apparatus attaches to the livestock through a pair of elastic and adjustable straps. Each elastic strap crosses over a forelimb armpit and over the back of the livestock before securing to the apparatus. In some embodiments, the apparatus may have a bottom surface with an uneven ridges for frictionally engaging the livestock and further minimizing slippage.

This disposition of the apparatus helps ensure retention of the apparatus directly above, or adjacent to the withers, such as on the median line of the livestock over the vertebral column. Additionally, since the apparatus positions directly above the median line of the vertebral column, rather than the sides, front, or rear of the livestock; accidental engagements with other livestock, trees, fences, and scratching posts is minimized. Furthermore, the disposition of the apparatus on the back of the livestock does not interfere with free walking, lying down, eating, drinking, or mating by the livestock.

In some embodiments, the apparatus may be powered by a solar cell. The solar cell provides renewable energy for recharging a power source in the apparatus. The solar cell is configured to orient upwardly relative to the back of the livestock to minimize damage from other livestock and objects, and optimize the capturing of sunlight. The use of the solar cell allows the apparatus to function for long periods of time without intervention. Those skilled in the art will recognize that livestock on large ranches may travel long distances for long durations. The use of renewable energy, such as that provided by a solar cell helps reduce maintenance costs for the apparatus and method, as a whole.

In some embodiments, the apparatus may include at least one reservoir for containing various types of medication. The use of multiple reservoirs enables a variety of medications to be administered to the livestock at periodic intervals. The medications are those generally used for the treatment of livestock, which may include, without limitation, tick repellant, pest repellant, antibiotics, and deworming agents.

The medication is administered by the apparatus through a dispenser. The dispenser operatively connects to each reservoir. The dispenser may utilize various means to dispense the medication from the at least one reservoir, depending on the form of the medication. The at least one dispenser may dispense the medication topically on the wither area of the livestock, including intravenous, subcutaneous, subcutaneous implants, intramuscular, and intraperitoneal. In one embodiment, the dispenser may spray or stream a liquid medication, or allow a foam to dribble onto the livestock. The dispenser may also inject the medication, as needed. Though, in most embodiments, the dispenser sprays a liquid repellant or deworming agent directly on the wither area of the livestock, whereby the medication is absorbed into the bloodstream through the skin, dispersed on the skin or vaporized over the skin.

In some embodiments, the apparatus may include a tracking portion that tracks both the position of the livestock in a predetermined area, and the orientation of the livestock. The position of the livestock may be useful for herd management. For example, monitoring a stray animal from the herd. The tracking portion may utilize any tracking apparatus known in the art to monitor the position of the livestock, including, without limitation, GPS, Wi-Fi system, and radio frequency instruments. A tracking processor, a tracking chip, and an antenna may be used inside the apparatus to operate the tracking portion. Similarly to the position of the livestock, the orientation of the livestock may be useful for determining the effects of the medication and the general health of the livestock. For example, a livestock that is oriented on its side may indicate a sick or dead livestock. A gyroscope and various modifications thereof may be effective for indicating the orientation of the livestock.

In some embodiments, the apparatus and method enables both direct and remote operation and monitoring of the apparatus. The apparatus may be operated directly through a control portion, which includes a display screen and various buttons and switches. The control portion enables direct functional operation of the apparatus, including, without limitation, powering on and off the apparatus, adjusting the amount of medication that is administered by the dispenser, switching between medications in different reservoirs, setting the sensitivity of the tracking portion, adjusting the parameters of a tracking range, and setting alarms to indicate various events. The display screen serves to display the results of these functions, including data that indicates the intervals at which the apparatus releases the medication. In one embodiment, the apparatus may also be controlled through a remote processor, whereby generally the same functions are possible as with the more directly operated control portion on the apparatus. In another embodiment, an alarm on the apparatus indicates to the remote processor when the administration of the medication is flawed or nonoperational.

One objective of the present invention is to provide an apparatus that directionally dispenses medication directly onto livestock or onto any quadruped.

Another objective is to provide at least one strap that holds the apparatus in a position on the withers, such that the apparatus is not disturbed by inadvertent engagement with other livestock or objects.

Another objective is to provide a dispenser that can be oriented to directionally administer the medication on the livestock.

Yet another objective is to provide an apparatus that can hold more than one type of medication.

Yet another objective is to monitor the administration of the medication and alert when the administration is flawed.

Yet another objective is to monitor the orientation of the livestock to determine if the livestock is sick or dead.

Yet another objective is to power the apparatus with a renewable energy source, such as solar energy.

Yet another objective is to provide medication dispenser that attaches directly on livestock and that requires minimal time and effort to operate.

Yet another objective is to provide an inexpensive to implement system and method that is adaptable across a variety of livestock.

### DRAWINGS

These and other features, aspects, and advantages of the present invention will become better understood with regard to the following description, appended claims, and drawings where:

FIG. 1 is a flowchart diagram of an exemplary method for managing livestock;

FIG. 2 is a perspective view of an exemplary livestock receiving medication from an attached exemplary apparatus for managing livestock;

FIG. 3 is a perspective view of an exemplary apparatus for managing livestock; and

FIG. 4 a block diagram depicting an exemplary client/server system which may be used by an exemplary web-enabled/networked embodiment of the present invention.

### DESCRIPTION

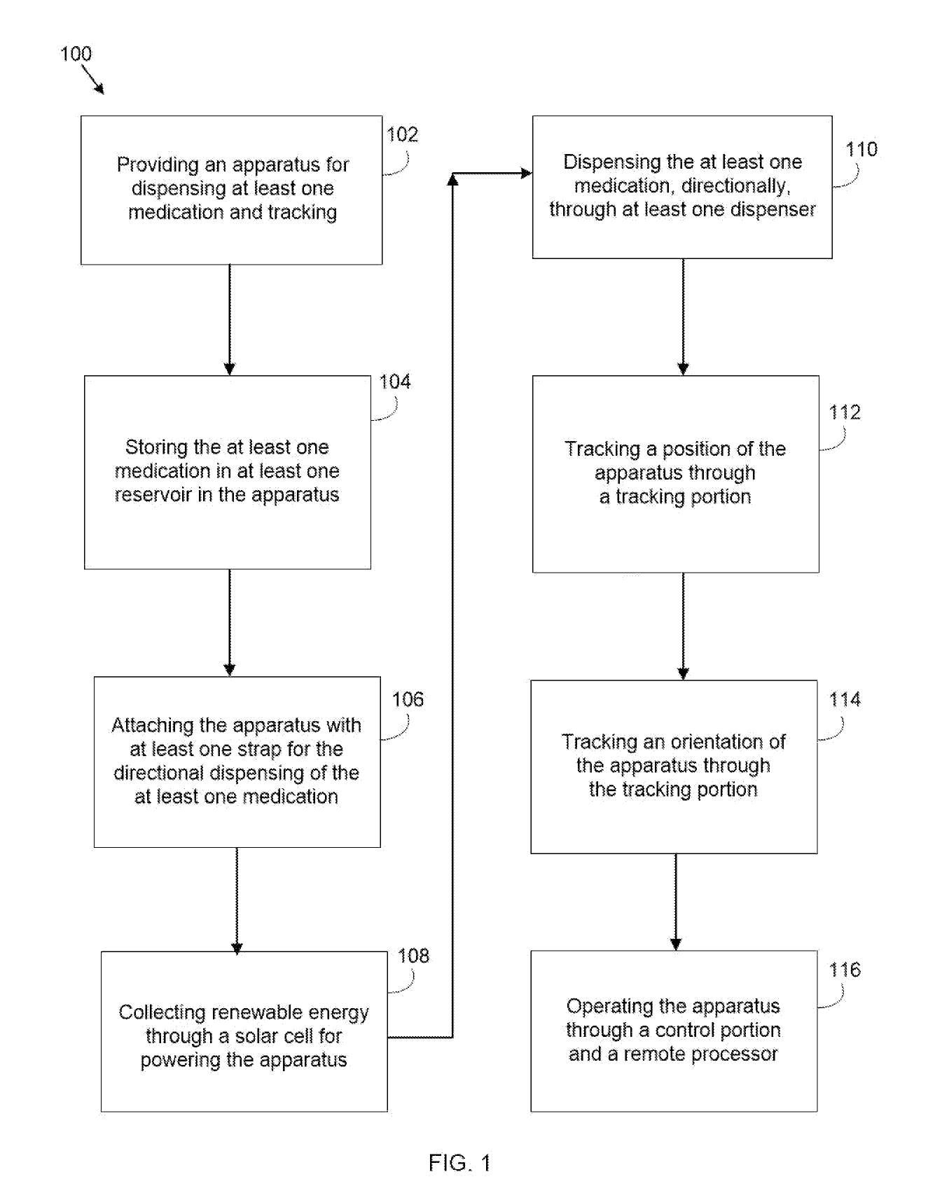

One embodiment, referenced in FIGS. 1-4, illustrates a method 100 and apparatus 200 for managing livestock 300 through the administration of at least one medication 306 and tracking the position and orientation of at least one livestock 300. The livestock 300 may include, without limitation, bovines, sheep, goats, horses, chickens, and wild animals. The method 100 and apparatus 200 may be utilized with a single animal, a herd of livestock 300, or a herd leader, such as a bull. The method 100 and apparatus 200 may be operated and tracked directly on the livestock 300 through a control portion 212, or remotely through a remote processor 214 that operatively connects to the control portion 212. The at least one medication 306 may include a topically administered vaccine, antibiotic, or tick repellant known in the art to treat and fortify livestock 300. Though, additional embodiments of the method 100 and apparatus 200 are discussed below.

FIG. 1 illustrates a flowchart diagram of an exemplary method 100 for managing livestock 300. The method 100 automatically administers medication 306 to at least one livestock 300 while also monitoring the effect of the medication 306, and tracking the movement and orientation of the livestock 300. The method 100 provides an apparatus 200 that periodically administers the at least one medication 306 at a predetermined dosage. Additionally, the method 100 uses the apparatus 200 to track the position and orientation of the livestock 300 to help determine whether the medication 306 is effective. The method 100 minimizes time and costs associated with managing livestock 300, especially over large areas or with large numbers of livestock 300.

In some embodiments, the method 100 may include an initial Step 102 of providing an apparatus 200 for directionally administering at least one medication 306 and tracking functions. The apparatus 200 is defined by a housing 216 having a power reception panel, an engagement panel, and a plurality of sidewalls. The housing 216 may include a cube shape, a rectangular shape, or an elongated planar shape. In any case, the housing 216 is configured to rest comfortably on the wither area 302 of the livestock 300. The engagement panel may include an uneven surface, such as ridged or textured characteristics that inhibit slippage from the skin and fur of the livestock 300. In one embodiment, an adhesive may be used on the engagement panel to restrict slippage and movement of the apparatus 200. The power reception panel may be configured to support a solar cell 204. The power reception panel is generally oriented upwardly, so as to enable the solar cell 204 to generally face the sun.

The method 100 may further comprise a Step 104 of storing the at least one medication 306 in at least one reservoir 208a, 208b in the apparatus 200. The medication 306 is that generally used to treat or fortify livestock 300. The apparatus 200 may store more than one type of medication 306 for periodic administration thereof. The use of multiple reservoirs 208a, 208b enables a variety of medications 306 to be administered to the livestock 300 at periodic intervals. The at least one medication 306 may include, without limitation, tick repellant, pest repellant, antibiotics, deworming agents, Levamisole, and Furosemide, and Equine chorionic gonadotropin. The medication 306 may be effective in preventing infection from common pests that are dangerous to livestock 300, including, without limitation, horn flies, Haematobia irritans; face flies, Musca autumnalis De Geer; the Gulf Coast tick, Amblyomma maculatum Koch; the screwworm, Cochliomyia hominivorax; and the southern cattle tick, Ripicephalus (Boophilus) microplus.

A Step 106 includes attaching the apparatus 200 with at least one strap 202 for directional administration of the at least one medication 306. The apparatus 200 utilizes at least one strap 202 to attach proximally to the wither 302 of the livestock 300 in a disposition that minimizes movement and slippage by the apparatus 200, but also optimizes directional administration of the medication 306. In one embodiment, the apparatus 200 attaches to the livestock 300 through a pair of elastic and adjustable straps 202. Each strap 202 crosses over a forelimb armpit and over the back of the livestock 300 before securing to the apparatus 200. In one embodiment, the strap 202 may include a leather strap 202 having buckles and multiple free ends for securing to the livestock 300. The at least one strap 202 may also support a pair of sleeves that receive the limbs 304 of the livestock 300. The strap 202 extends from the sleeves to the apparatus 200. In addition to the strap 202, the engagement panel of the apparatus 200 may have uneven ridges for frictionally engaging the livestock 300 and further minimizing slippage.

This disposition of the apparatus 200 helps ensure retention of the apparatus 200 directly above, or adjacent to the wither 302. This can include the median line of the livestock 300 over the vertebral column. Additionally, since the apparatus 200 positions directly above the median line of the vertebral column, rather than the sides, front, or rear of the livestock 300; accidental engagements with other animals, trees, fences, and scratching posts is minimized. Furthermore, the disposition of the apparatus 200 on the back of the livestock 300 does not interfere with free walking, lying down, eating, drinking, or mating by the livestock 300.

In some embodiments, a Step 108 comprises collecting renewable energy through a solar cell 204 for powering the apparatus 200. In some embodiments, the apparatus 200 may be powered by a portable solar cell 204 that integrates into the apparatus 200 from the power reception panel. The solar cell 204 provides renewable energy for recharging a power source, such as a battery, in the apparatus 200. The solar cell 204 is configured to orient upwardly relative to the back of the livestock 300 to minimize damage from other livestock 300 and objects, and optimize the capturing of sunlight. The use of the solar cell 204 allows the apparatus 200 to function for long periods of time without intervention.

A Step 110 includes dispensing the at least one medication 306 through at least one dispenser 206. The medication 306 is administered periodically and at predetermined dosages through the at least one dispenser 206. Each dispenser 206 operatively connects to each reservoir 208a, 208b of medication 306, such that multiple dispensers 206 can be used. The dispenser 206 may be adjustable for directional administration of the mediation 306. The periodic administration may be set at fixed time points or can vary along the year depending on the capabilities of the control portion 212 and the processor container therein.

Those skilled in the art will recognize that the wither 302 is a prime location for medication 306 to be absorbed topically into the bloodstream. The dispenser 206 may utilize various means to dispense the medication 306 on the wither 302 from the at least one reservoir 208a, 208b, depending on the form of the medication 306. For example, the medication 306 is administered by the at least one dispenser 206 through topical means, such as spraying, pouring, and dribbling on the fur and skin. In some embodiments, the at least one dispenser 206 may dispense the medication 306 through various topical means, including, without limitation, intravenous, subcutaneous, subcutaneous implants, intramuscular, and intraperitoneal. The at least one dispenser 206 sprays, pours, or dribbles the medication 306 in these instances. In one alternative embodiment, the at least one dispenser 206 utilizes an extendable tube to administer the medication 306 on the limbs 304 of the livestock 300. For example, the posterior end of the extended tube is fixed to the dorsal face of the croup by a string joined to the base of the tail.

In some embodiments, a Step 112 may include tracking a position of the apparatus 200 through a tracking portion 210. The apparatus 200 may include a tracking portion 210 that tracks both the position of the livestock 300 in a predetermined area, and the orientation of the livestock 300. The position of the livestock 300 may be useful for herd management. For example, monitoring a stray animal from the herd, or monitoring a herd that has broken through a fence. The tracking portion 210 may utilize any tracking apparatus 200 known in the art to monitor the position of the livestock 300, including, without limitation, a global positioning system, Wi-Fi system, and radio frequency instruments. A tracking processor, a tracking chip, and an antenna may be integrated with the tracking portion 210 to enhance the tracking function. In one embodiment, a smart phone may be operable to download a software application that enables tracking and other general functions of the apparatus 200.

A Step 114 comprises tracking an orientation of the apparatus 200 through the tracking portion 210. The tracking portion 210 is also used to monitor the orientation of the livestock 300. It is significant to note that awareness of the orientation of the livestock 300 (through the tracking portion 210) may be useful for determining the effects of the medication 306 and the general health of the livestock 300. For example, a livestock 300 that is oriented on its side may indicate a sick or dead livestock 300. A livestock 300 that repetitively turns in circles may be gnawing at a sore on the skin. The medication 306 can be adjusted to accommodate these potential ailments. The tracking portion 210 may use a gyroscope and various modifications thereof to indicate the orientation of the livestock 300.

A final Step 116 includes operating the apparatus 200 through a control portion 212 and a remote processor 214. The method 100 enables both direct and remote operation and monitoring of the apparatus 200. The apparatus 200 may be operated directly through a control portion 212, which includes a display screen, a map, and various buttons and switches. The control portion 212 enables direct functional operation of the apparatus 200, including, without limitation, powering on and off the apparatus 200, adjusting the amount of medication 306 that is administered by the dispenser 206, switching between medications 306 in different reservoirs 208a, 208b, setting the sensitivity of the tracking portion 210, adjusting the parameters of a tracking range, and setting alarms to indicate various events. The display screen serves to display the results of these functions, including data that indicates the intervals at which the apparatus 200 releases the medication 306. In one embodiment, the apparatus 200 may also be controlled through a remote processor 214; whereby generally the same functions are possible as with the more directly operated control portion 212 on the apparatus 200. The remote processor 214 may include, without limitation, a computer, a server, a database, a cloud, and a network. In another embodiment, an alarm (not shown) on the apparatus 200 indicates an event to the remote processor 214. The event may include, for example, when the administration of the medication 306 is flawed or nonoperational. Other events may include a low level of the at least one medication 306 in the at least one reservoir 208a, 208b, incorrect dosage amount, incorrect frequency of administration, misalignment of the dispenser 206, and dysfunction of the solar cell 204.

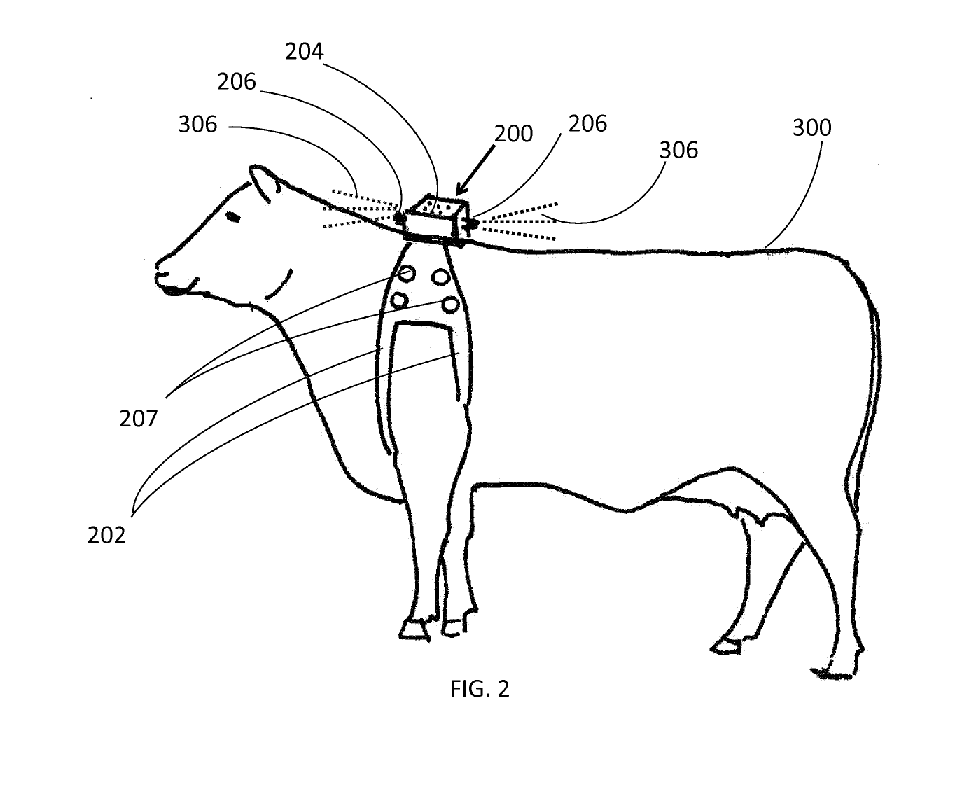

Turning now to FIG. 2, a livestock management apparatus 200 is illustrated. The apparatus 200 is defined by a housing 216 having a power reception panel, an engagement panel, and a plurality of sidewalls. The apparatus 200 mounts on an exemplary livestock 300, such as a bovine, goat, sheep, horse, or chicken. The apparatus 200 is configured to automatically administer at least one medication 306 to at least one livestock 300 while also monitoring the effect of the medication 306, and tracking the movement and orientation of the livestock 300. The medication 306 is administered periodically while the movement of the livestock 300 serves as an indication of whether the medication 306 is effective. The apparatus 200 is configured to minimize time and costs associated with managing livestock 300, especially over large areas or with large numbers of livestock 300.

In some embodiments, the apparatus 200 provides an administration and tracking means that attaches directly to the livestock 300. The apparatus 200 utilizes at least one strap 202 to attach proximally to the wither 302 of the livestock 300 in a disposition that minimizes movement and slippage by the apparatus 200, but also optimizes directional administration of the medication 306. In one embodiment, the apparatus 200 attaches to the livestock 300 through a pair of elastic and adjustable straps 202. Each elastic strap 202 crosses over a forelimb armpit and over the back of the livestock 300 before securing to the apparatus 200. In some embodiments, the apparatus 200 may have an engagement panel with an uneven ridges for frictionally engaging the livestock 300 and further minimizing slippage by the apparatus 200.

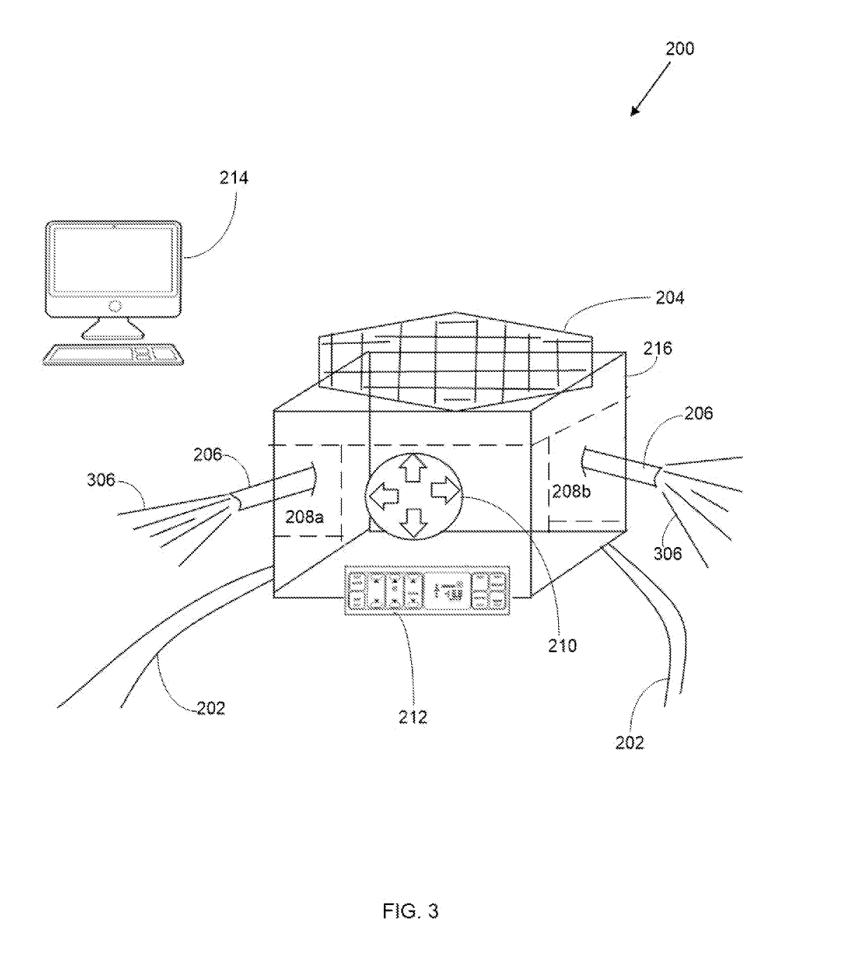

As referenced in FIGS. 2-3, the disposition of the apparatus 200 on the wither 302 helps ensure retention of the apparatus 200 directly above, or adjacent to the wither 302. The apparatus 200 positions directly above the median line of the vertebral column, rather than the sides, front, or rear of the livestock 300. This helps prevent accidental engagements with other animals, trees, fences, and scratching posts. Furthermore, the disposition of the apparatus 200 on the back of the livestock 300 does not interfere with free walking, lying down, eating, drinking, or mating by the livestock 300.

In a preferred embodiment, as shown in FIG. 2, at least one sensor 207 is attached to each strap 202 and is connected via wires or any other means of connection to the apparatus 200. The sensors 207 are placed on the straps 202, for when cattle are affected by flies or mosquitoes they get irritated and usually they use their tails and heads with a movement to repel these insects. Some devices such as ear tags and tale strips take advantage of this pattern behavior. These devices are impregnated with an insecticide so when cattle move their heads towards the flanks to repel the flies, part of the impregnated insecticide over the surface of the tag is passed onto the flanks or withers of the animal.

In this particular embodiment, the device takes advantage of the movement of the head to repel flies but in a different way. When the animal twists or moves the head to the flanks the head will touch the area of the withers where one or a series of pressure sensors or switches will trigger the spraying or delivery mechanism and the animal will get treated. Instead of a predetermined or set time for treatment, this will be accommodated to the animal's need and degree of irritation with a resulting optimization of the drug application. This can be used to control certain pests such as horn flies, Haematobia irritans (Linnaeus, 1758) and may be other flies and mosquitoes. Other pests will certainly require of a time frame predetermined treatment with fixed treatment time intervals such as ticks.

The use of a spray (instead of a pour-on or spot-on) has the advantage, in this particular embodiment, that the repellent or insecticide with have more chances to get to the front and rear legs, where mosquitos and some flies usually bite, if sprayed backwards. The front legs can easily be covered in this system by the use of spray, pour-on or spot-on released down or to the sides of the device as the product will pour down to the withers and to the front legs. The backward spray will get to the end of the animal and will then have an effect on the rear legs.

As shown in FIG. 3, the apparatus 200 may be powered by a solar cell 204. The solar cell 204 provides renewable energy for recharging a power source in the apparatus 200. The solar cell 204 is configured to orient upwardly relative to the back of the livestock 300 to minimize damage from other livestock 300 and objects, and optimize the capturing of sunlight. The use of the solar cell 204 allows the apparatus 200 to function for long periods of time without intervention.

In some embodiments, the apparatus 200 may include at least one reservoir 208a, 208b for containing various types of medication 306. The use of multiple reservoirs 208a, 208b enables a variety of medications 306 to be administered to the livestock 300 at periodic intervals. The medications 306 are those generally used for the treatment of livestock 300, which may include, without limitation, tick repellant, pest repellant, antibiotics, and deworming agents.

The medication 306 is administered by the apparatus 200 through at least one dispenser 206. The dispenser 206 operatively connects to each reservoir 208a, 208b. The dispenser 206 may utilize various means to dispense the medication 306 from the at least one reservoir 208a, 208b, depending on the form of the medication 306. The dispenser 206 is directionally adjustable. In one embodiment, the dispenser 206 may spray or stream a liquid medication 306, or allow a foam to dribble onto the livestock 300. The dispenser 206 may also inject the medication 306, as needed. Though, in most embodiments, the dispenser 206 sprays a liquid repellant or deworming agent directly to the wither 302 area of the livestock 300, whereby the medication 306 is absorbed into the bloodstream through the skin.

In some embodiments, the apparatus 200 may include a tracking portion 210 that tracks both the position of the livestock 300 in a predetermined area, and the orientation of the livestock 300. The position of the livestock 300 may be useful for herd management. A tracking processor, a tracking chip, and an antenna may be used inside the apparatus 200 to operate the tracking portion 210. Similarly to the position of the livestock 300, the orientation of the livestock 300 may be useful for determining the effects of the medication 306 and the general health of the livestock 300. A gyroscope may be effective for indicating the orientation of the livestock 300.

In some embodiments, the apparatus 200 may be operated both directly and remotely. The apparatus 200 is operated directly through a control portion 212, which includes a display screen and various buttons and switches. The control portion 212 enables direct functional operation of the apparatus 200. The apparatus 200 can also be controlled through a remote processor 214. The remote processor 214 may include a computer, a server, a cloud, and a network. The remote processor 214 enables a remote livestock manager to both monitor the herd, and operate the apparatus 200.

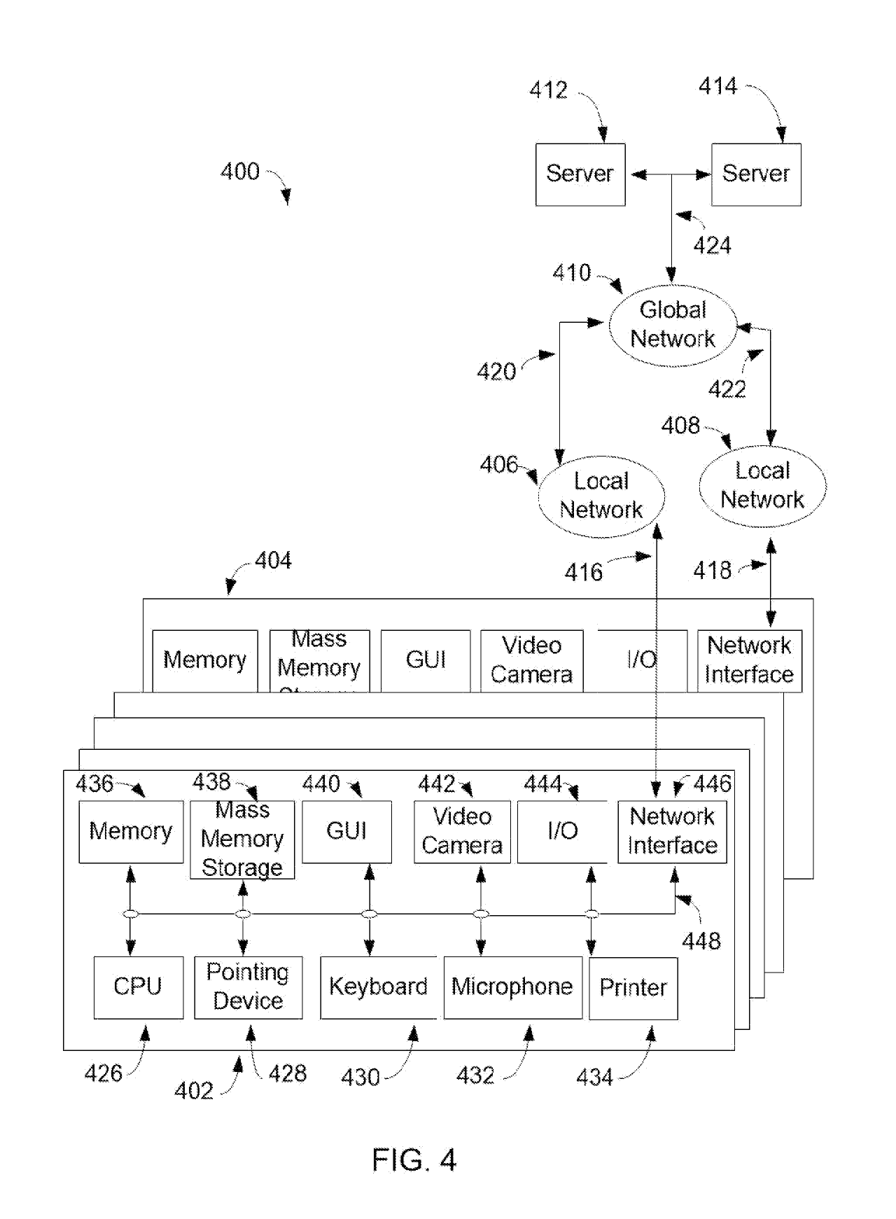

FIG. 4 is a block diagram depicting an exemplary client/server system which may be used by an exemplary web-enabled/networked embodiment of the present invention.

A communication system 400 includes a multiplicity of clients with a sampling of clients denoted as a client 402 and a client 404, a multiplicity of local networks with a sampling of networks denoted as a local network 406 and a local network 408, a global network 410 and a multiplicity of servers with a sampling of servers denoted as a server 412 and a server 414.

Client 402 may communicate bi-directionally with local network 406 via a communication channel 416. Client 404 may communicate bi-directionally with local network 408 via a communication channel 418. Local network 406 may communicate bi-directionally with global network 410 via a communication channel 420. Local network 408 may communicate bi-directionally with global network 410 via a communication channel 422. Global network 410 may communicate bi-directionally with server 412 and server 414 via a communication channel 424. Server 412 and server 414 may communicate bi-directionally with each other via communication channel 424. Furthermore, clients 402, 404, local networks 406, 408, global network 410 and servers 412, 414 may each communicate bi-directionally with each other.

In one embodiment, global network 410 may operate as the Internet. It will be understood by those skilled in the art that communication system 400 may take many different forms. Non-limiting examples of forms for communication system 400 include local area networks (LANs), wide area networks (WANs), wired telephone networks, wireless networks, or any other network supporting data communication between respective entities.

Clients 402 and 404 may take many different forms. Non-limiting examples of clients 402 and 404 include personal computers, personal digital assistants (PDAs), cellular phones and smartphones.

Client 402 includes a CPU 426, a pointing device 428, a keyboard 430, a microphone 432, a printer 434, a memory 436, a mass memory storage 438, a GUI 440, a video camera 442, an input/output interface 444 and a network interface 446.

CPU 426, pointing device 428, keyboard 430, microphone 432, printer 434, memory 436, mass memory storage 438, GUI 440, video camera 442, input/output interface 444 and network interface 446 may communicate in a unidirectional manner or a bi-directional manner with each other via a communication channel 448. Communication channel 448 may be configured as a single communication channel or a multiplicity of communication channels.

CPU 426 may be comprised of a single processor or multiple processors. CPU 426 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 436 is used typically to transfer data and instructions to CPU 426 in a bi-directional manner. Memory 436, as discussed previously, may include any suitable computer-readable media, intended for data storage, such as those described above excluding any wired or wireless transmissions unless specifically noted. Mass memory storage 438 may also be coupled bi-directionally to CPU 426 and provides additional data storage capacity and may include any of the computer-readable media described above. Mass memory storage 438 may be used to store programs, data and the like and is typically a secondary storage medium such as a hard disk. It will be appreciated that the information retained within mass memory storage 438, may, in appropriate cases, be incorporated in standard fashion as part of memory 436 as virtual memory.

CPU 426 may be coupled to GUI 440. GUI 440 enables a user to view the operation of computer operating system and software. CPU 426 may be coupled to pointing device 428. Non-limiting examples of pointing device 428 include computer mouse, trackball and touchpad. Pointing device 428 enables a user with the capability to maneuver a computer cursor about the viewing area of GUI 440 and select areas or features in the viewing area of GUI 440. CPU 426 may be coupled to keyboard 430. Keyboard 430 enables a user with the capability to input alphanumeric textual information to CPU 426. CPU 426 may be coupled to microphone 432. Microphone 432 enables audio produced by a user to be recorded, processed and communicated by CPU 426. CPU 426 may be connected to printer 434. Printer 434 enables a user with the capability to print information to a sheet of paper. CPU 426 may be connected to video camera 442. Video camera 442 enables video produced or captured by user to be recorded, processed and communicated by CPU 426.

CPU 426 may also be coupled to input/output interface 444 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 426 optionally may be coupled to network interface 446 which enables communication with an external device such as a database or a computer or telecommunications or internet network using an external connection shown generally as communication channel 416, which may be implemented as a hardwired or wireless communications link using suitable conventional technologies. With such a connection, CPU 426 might receive information from the network, or might output information to a network in the course of performing the method steps described in the teachings of the present invention.

While the inventor's above description contains many specificities, these should not be construed as limitations on the scope, but rather as an exemplification of several preferred embodiments thereof. Many other variations are possible. For example, the shape and dimensions of the dispenser 206 may vary without affecting the objective of the apparatus 200. Accordingly, the scope should be determined not by the embodiments illustrated, but by the appended claims and their legal equivalents.

What is claimed is:

1. An apparatus for managing livestock through the administration of medication and tracking, the apparatus comprising:
a housing defined by a power reception panel, an engagement panel, and a plurality of sidewalls,
wherein the engagement panel has a substantially uneven surface;
at least one strap having a generally elongated disposition;
a solar cell disposed to couple to the power reception panel, the solar cell configured to generate renewable energy;
at least one reservoir defined by a space for storing at least one medication;
at least one dispenser defined by a channel for directional dispensing the at least one medication;
at least one tracking portion configured to track a position and an orientation of the apparatus; and
a control portion operatively connected to the at least one dispenser and the at least one tracking portion.

2. The apparatus of claim 1, wherein the apparatus is operatively attached to the at least one livestock or a quadruped.

3. The apparatus of claim 2, wherein the power reception panel is disposed to orient upwardly, towards the sun.

4. The apparatus of claim 3, wherein the solar cell is configured to recharge a battery in the apparatus.

5. The apparatus of claim 4, wherein the uneven surface of the engagement panel is defined by a ridged texture configured to create frictional attachment with the skin of the at least one livestock for inhibiting slippage of the apparatus.

6. The apparatus of claim 5, wherein the apparatus is disposed directly on, or adjacently to the wither of the at least one livestock.

7. The apparatus of claim 6, wherein the at least one strap is configured to fasten the apparatus to the at least one livestock.

8. The apparatus of claim 7, wherein the at least one strap is arranged to cross over a forelimb armpit and over the back of the at least one livestock.

9. The apparatus of claim 8, further comprises at least one sensor that is attached to each strap and is connected to the apparatus.

10. The apparatus of claim 9, wherein the at least one dispenser administers the at least one medication topically.

11. The apparatus of claim 10, wherein the at least one dispenser is a directionally adjustable spray tube.

12. The apparatus of claim 11, wherein the at least one dispenser is configured to extend for administering the at least one medication on the limbs of the at least one livestock.

13. The apparatus of claim 1, wherein the at least one medication includes at least one member selected from the group consisting of: tick repellant, pest repellant, antibiotics, deworming agents, or hormones.

14. The apparatus of claim 1, wherein the control portion includes at least one member selected from the group consisting of: a button, a switch, a display screen, an antenna, and an alarm.

15. The apparatus of claim 1, further comprises at least one sensor that is attached to each strap and is connected to the apparatus.

16. The apparatus of claim 1, wherein the tracking portion is configured to track the position of the apparatus with a global positioning system or a radio frequency system.

17. The apparatus of claim 1, wherein the tracking portion tracks the orientation of the apparatus with a gyroscope.

18. The apparatus of claim 1, further including a remote processor that is operatively connected to the control portion.

19. A method for managing livestock through the administration of medication and tracking, the method comprising:
providing an apparatus for dispensing at least one medication and tracking;
storing the at least one medication in at least one reservoir in the apparatus;
attaching the apparatus with at least one strap for the directional dispensing of the at least one medication;
collecting renewable energy through a solar cell for powering the apparatus;
dispensing the at least one medication, directionally, through at least one dispenser;
tracking a position of the apparatus through a tracking portion;
tracking an orientation of the apparatus through the tracking portion; and
operating the apparatus through a control portion and a remote processor.

20. The method of claim 19, wherein the method further includes a step of indicating an event from the apparatus with an alarm.
